(19) United States
(12) Patent Application Publication
(10) Pub. No.: US 2005/0261561 A1
(43) Pub. Date: Nov. 24, 2005

(54) Blood testing and therapeutic compound delivery system

(75) Inventors: Christopher W. Jones, South Jordan, UT (US); Carl Embry, Boulder, CO (US); Allen Tracy, Erie, CO (US)

Correspondence Address: THORPE NORTH & WESTERN, LLP., 8180 SOUTH 700 EAST, SUITE 200, P.O. BOX 1219, SANDY, UT, 84070, US

(73) Assignee: Christopher W. Jones

(21) Appl. No.: 11/134,221

(22) Filed: May 20, 2005

Related U.S. Application Data
(60) Provisional application No. 60/573,888, filed on May 24, 2004.

(51) Int. Cl. A61K 38/28; A61B 5/00

(52) U.S. Cl. 600/315

(57) ABSTRACT

A system and method are provided for determining intravenous blood levels of a target compound contained in a blood vessel of a patient. The method includes the operation of detecting concentrations of the target compound within a patient's blood using a sensor device configured to optically test blood at a location within the blood vessel. Another operation is calculating a measured amount of a therapeutic compound to administer into the patient's bloodstream based on the concentrations of the target compound in the blood. The measured amount of therapeutic compound may then be pumped through the catheter into the patient's blood.

### CROSS-REFERENCE TO RELATED APPLICATIONS AND CLAIM OF PRIORITY

Priority of U.S. Provisional patent application Ser. No. 60/573,888 filed on May 24, 2004 is claimed.

### FIELD OF THE INVENTION

The present invention relates generally to blood testing.

### BACKGROUND

Hyperglycemia as a result of diabetes mellitus is well known to be harmful if left under treated for long periods of time. Much effort has been devoted to simplifying and refining the treatment of ambulatory diabetic patients and their hyperglycemia. Attempts have been made to control blood sugar with a variety of external and implantable insulin pumps. Generally, people with diabetes need to check their blood sugar levels to be able to control such pumps effectively. The most widely employed technique for checking insulin levels is to use a small, sharp lancet that pierces the skin and subsequently a drop of blood is withdrawn and placed on a device for glucose measurement. The medical literature is replete with ideas to measure the sugar level with non-invasive ways. Progress in these areas is likely to help diabetics “stay in control” and prevent the harm that otherwise awaits them over time.

Traditionally, medical professionals have treated hospitalized diabetic patients differently. There has recently been a revolution in the treatment of diabetic and hyperglycemic patients in a hospitalized or critical care setting. Once the dictum was, “Keep 'em sweet.” Now there is proof of decreased mortality and morbidity when blood glucose levels are kept at or near normal range. A 1995 study by Malmberg showed a 29% relative risk reduction in death when diabetics with an acute myocardial infarction were treated with an intensive insulin regimen. In a surgical intensive care unit, Van den Berghe found a 34% decrease in mortality with an intensive insulin protocol to keep glucose readings between 80 and 110 mg/dl. Finney studied medical and surgical patients in an intensive care unit and showed that tight glucose control (under 145 mg/dl) rather than insulin treatment was accountable for saving lives.

It is interesting that the decreased death rate has been found not only in different populations of diabetics but in non-diabetics too. The latter two studies enrolled all patients in an Intensive Care Unit (ICU) with hyperglycemia. In one study, only thirteen percent of patients had a previous diagnosis of diabetes. Additionally, a number of secondary endpoints were monitored which showed an impressive decrease in morbidity. In the intensive insulin therapy group, there were 46% less bloodstream infections, 41% less complete renal failure, 50% less red blood cell transfusions and 44% less critical care polyneuropathy. Experts in the field are calling for intensive insulin therapy to maintain strict glucose control in most patients with diabetes and/or hyperglycemia while hospitalized. Keeping glucose levels “tightly controlled” means defining a range of glucose concentration within the blood which is normal or near normal, then working to prevent fluctuations away from this range. Work is an operative word here because it is very difficult to achieve this goal. Physicians, nurses, pharmacists, laboratory technicians, nutritionists and others put forth great effort for many of the patients with hyperglycemia. These efforts, though laudable, are frequently unsuccessful and are always expensive. Sometimes in a postoperative patient, a surgeon will call in an internist or an endocrinologist solely to manage the blood glucose levels. Whenever there are large fluctuations in the glucose levels, blood is drawn hourly instead of every 4 or 6 hours. This is done because more frequent blood glucose testing allows for more responsive insulin infusion changes and thus “tighter control.” This does incur more patient discomfort, more nursing and lab time spent, and more expense.

### SUMMARY OF THE INVENTION

The invention provides a system and method for determining intravenous blood levels of a target compound contained in a blood vessel of a patient. The method includes the operation of detecting concentrations of the target compound within a patient's blood using a sensor device configured to optically test blood at a location within the blood vessel. Another operation is calculating a measured amount of a therapeutic compound to administer into the patient's bloodstream based on the concentrations of the target compound in the blood. The measured amount of therapeutic compound may then be pumped through the catheter into the patient's blood.

Additional embodiments of the invention will be apparent from the detailed description which follows, taken in conjunction with the accompanying drawings, which together illustrate, by way of example, features of the invention.

### BRIEF DESCRIPTION OF THE DRAWINGS

FIG. 1 illustrates a cross-sectional view of a catheter inserted under the skin with catheter tubing and fiber optic cable in accordance with an embodiment of the present invention;

FIG. 2 is front view of an embodiment of a housing illustrating certain elements of a bloodstream detection and insulin delivery system; and

FIG. 3 is a flow chart illustrating an embodiment of method for determining blood levels of a target compound as measured from within a blood vessel of a patient.

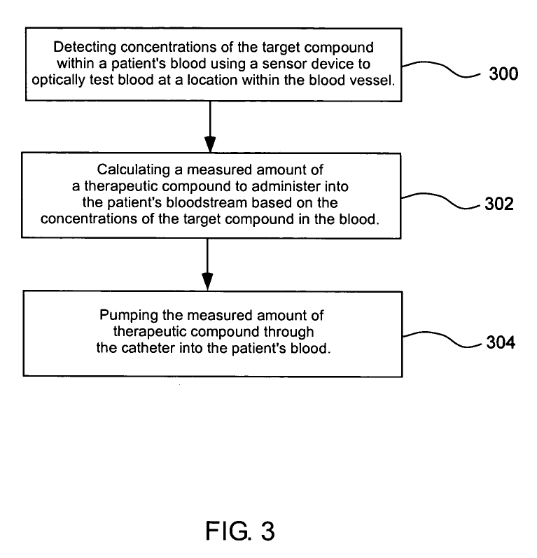

### DETAILED DESCRIPTION

Reference will now be made to the exemplary embodiments illustrated in the drawings, and specific language will be used herein to describe the same. It will nevertheless be understood that no limitation of the scope of the invention is thereby intended. Alterations and further modifications of the inventive features illustrated herein, and additional applications of the principles of the inventions as illustrated herein, which would occur to one skilled in the relevant art and having possession of this disclosure, are to be considered within the scope of the invention.

The system and method of the present invention is designed to overcome the obstacles and expenses described previously while delivering “tighter” blood glucose control than can be performed otherwise, thereby decreasing the mortality and morbidity of in-hospital patients. In particular, discomfort, time and expense are all minimized in this invention which controls blood glucose levels with an automated, close-loop system.

The present system and method can test and record blood glucose concentration nearly continuously or at short intervals, and then use the test information to deliver insulin at defined time intervals. For example, the insulin can be delivered at one-minute intervals. The delivery can be accomplished with the placement of at least one intravenous (IV) catheter and the attachment of the insulin delivery system to that catheter. Even though the present invention provides automated treatment, many safety and nursing intervention features can be included in the system.

The present invention relates to a system of glucose level sensing and insulin delivery designed to maintain blood glucose readings within a very narrow range with minimal human effort. Intravenous methods are employed for both testing and delivery. Particularly, the present invention employs glucose testing in the bloodstream along with an insulin delivery mechanism that can respond to the amounts of glucose measured and then deliver insulin solutions directly into the bloodstream.

The present invention helps overcome the problems associated with blood draws or long delays between glucose readings. Another beneficial aspect is that the amount of patient grimacing and rushing of nurses can be minimized. In addition, the consulting physician may be spared time and the patient and the patient's family may be spared extra cost. Most importantly will be the one in nine patients whose life is spared because of the present invention.

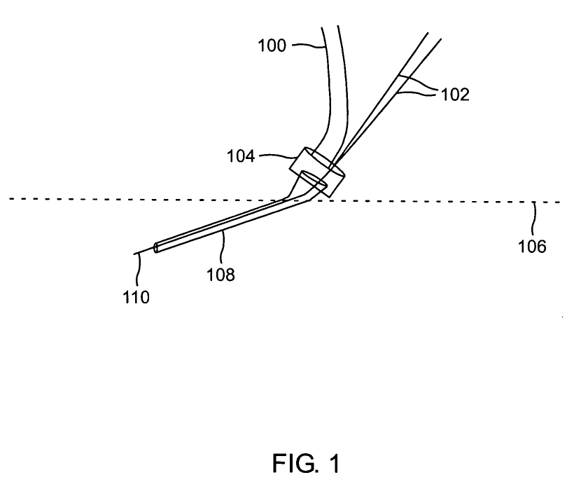

One embodiment of the system can include elements that enable substantially continuous blood glucose monitoring and provide the delivery of controlled amounts of insulin in response to the glucose level that is measured. FIG. 1 illustrates a cross-sectional view of a catheter 104 inserted under the skin 106 with catheter tubing 100 and fiber optic cable 102 in accordance with an embodiment of the present invention. Glucose levels can be directly measured within a blood vessel via the insertion of the fiber optic cable that passes through the IV catheter 108 and into the blood. In addition, the fiber optic cable can be secured to the IV catheter. The free end of the fiber optic cable 110 may be configured to extend a small distance beyond the catheter end in order to be constantly bathed in flowing blood. For example, in one embodiment an end of the fiber optic cable can be placed through the catheter, exit the catheter, and protrude roughly one third of a centimeter into the blood vessel.

Alternatively, the fiber optic cable can be located either flush with or just inside the catheter at a location that allows the optical measurements in the blood to take place. Another embodiment is for the IV catheter to be a central catheter with any number of lumens wherein the fiber optic cable can be inserted through the hollow portion of the catheter tube or a central catheter that already contains a fiber optic cable within the catheter wall structure. The portion of the fiber optic cable extending into the blood can be coated with an anticoagulant to avoid the problem of potential thrombosis within a blood vessel.

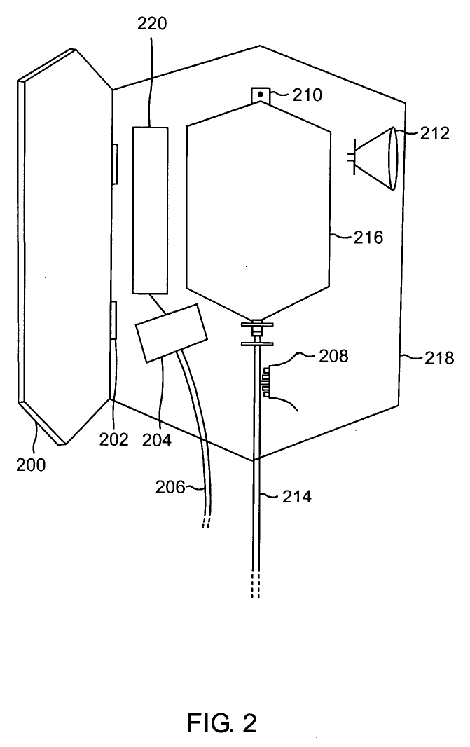

FIG. 2 is front view of an embodiment of a housing illustrating certain elements of an in the bloodstream detection and insulin delivery system. A laser device 204 coupled to the fiber optic cable 206 can provide a source for electromagnetic energy or [symbol] light [symbol] that leaves the end of the cable and reflects off the glucose within the blood. This reflected light can be captured by the end of the fiber optic cable or by a second fiber optic cable and returned to a receiver or sensor 204. The receiver can be a diode type receiver.

In the example of a single fiber optic cable, the backscatter from the pulse of electromagnetic energy can be captured as it reflects from the glucose within the blood and directs optical or electromagnetic energy back to the laser device. Alternatively, a second fiber optic cable can capture the backscatter. The construction of the fiber optic cable will allow transference of electromagnetic energy bidirectionally. The amount of reflected light energy can be correlated with the amount of glucose in the blood. In another embodiment a second cable can extend beyond the first optical cable and be positioned to receive direct (not reflected or backscattered) optical signals from the first optical cable. Additional details of embodiments for fiber optic system will be discussed later.

An additional unit 220 or calculator module for the system in this embodiment uses the measured input from the return fiber optic cable and data input from a human source (e.g., a medical professional) to calculate an appropriate amount of insulin to deliver. Other information can be input into the electronic hardware and software with the goal of following designed calculation methods to determine how much of a given fluid should be pumped down the IV catheter tubing.

An interface with a medical professional may be provided on or with the housing face 200 to enable the input of information which can include but not be limited to: a subject's identifying feature (e.g. name), a subject's height, a subject's weight, and the blood glucose goal. In addition, through this interface a medical professional can respond to alarms or change default settings for the operation of the system. One embodiment of this interface may be a touch screen, or another embodiment may be a series of buttons. Other computing interfaces known to those skilled in the art may be used.

An insulin delivery mechanism can work in cooperation with the calculation module to deliver the appropriate amounts of insulin to the patient. A disposable bladder 216 containing a fluid with insulin can be used with a pump. For example, an insulin-saline mix may be used in the bladder. A pump 208 may be located directly in contact with or may be coupled with the IV tubing and can “force” or pump the appropriate amount of fluid down the IV tubing, through the IV catheter, and into the blood vessel. This insulin will react in the patient to lower glucose levels which will subsequently be measured by the optical detection portion of this system and drive additional insulin delivery as desired. The calculation module again receives this measurement and performs its function then drives the pumping mechanism thereby delivering the exact amount of insulin to keep blood glucose levels within a narrow range.

In one embodiment, a calculation in the calculation module determines how much the pumping plates are moved which thereby squeezes or pumps out a given amount of the insulin containing fluid down a tube. This catheter tube is generally hollow but the tube may contain the aforementioned fiber optic cable. However, the fiber optic cable may be located outside the catheter tube but be combined into the catheter's plastic over molding and/or needle. For example, the fiber optic cable(s) can be secured to a plastic cap that screws onto the IV catheter external to the patient's body thereby securing the position of the cables. Such a catheter configuration still has room to allow insulin solution flow. The combination of the tube and cable that are attached securely to the IV catheter allow the fluid to enter the blood vessel. The pumping mechanism can alternatively be any type of medical or insulin pump known to those skilled in the art.

Dual fiber optic cables 206 may run from the IV cap up to the container where the majority of the insulin storage, calculation, pumping, and delivery equipment is stored. As discussed, the fibers can run either within or without the hollow IV tubing 214. For example, the fiber optics can be completely separate from the catheter tubing except where the catheter comes in proximity to and enters into the catheter end.

As mentioned, the fiber optic cables 206 can enter the housing container 218 and connect with a laser generation device 204. The laser is a tunable laser that can be tuned to a selected wavelength. For example, the laser can be tuned to a wavelength related to glucose. This laser can emit a burst of electromagnetic energy at a given wavelength with enough power to return a pulse of light energy back up a fiber optic cable to a diode receiver. The electromagnetic energy from the laser travels along the cable then exits the cable at the free end. Generally speaking, the original pulse of energy scatters as it reflects off of the glucose. The free end of the return fiber optic cable or the second fiber optic cable can pick up this backscatter. The receiving diode can record the signal which may be translated to a glucose level measured in milligrams per deciliter.

The laser device can be set to deliver the energy for a pre-defined period (e.g., partial seconds, multiple seconds, or minutes) and thereby determine glucose concentrations in the blood via the fiber optic cables and the receiving diode. This measurement information is transferred and recorded using the appropriate computer hardware and software.

The intravenous monitoring system and method avoids many of the previous limitations associated with non-invasive optical measurement methods such as absorption and scatter by cutaneous and sub-cutaneous tissues. Chemical and fluorescent techniques are limited by the problems of time delay to equilibration of intravenous and interstitial glucose, fluid shifting in critically ill patients, and consumption of the chemical or fluorescent compound. These limitations are overcome by this invention because of direct blood measurements and the lack of chemical or fluorescent reaction. The implementation of the system via an intravenous fiber detection and delivery system also inflicts minimal trauma to the patient as there is a one-time insertion of the system that allows days or even weeks of accurate functioning.

The systems and methods for measuring blood glucose in the bloodstream will now be discussed in further detail. Particularly, the optical characteristics of coherent light emitted from a laser can be used for quantitative detection of glucose levels in the blood. All materials have a characteristic spectrographic signature that can be used for molecular identification. This signature can be based on either absorption or emission of light at different wavelengths. Associated with each spectral line is a line width, indicating an absorption band around the central wavelength. Associated with each material is an absorption parameter stated in units of cm⁻¹, that indicates the amount of absorption at a particular wavelength per unit length of the material. Therefore, when light at the appropriate absorption wavelength is transmitted through or reflected off a material that contains some amount of the target compound or molecule, then a detector comprised of a material sensitive at that wavelength can be used to provide a quantitative determination of the amount of the constituent contained in the subject material.

The absorptive characteristics of a material can be used by another technique to identify the target constituent content in a suspension volume. A laser is used to emit a narrow-band pulse at a wavelength known to be absorptive by a certain percentage in the target molecule. The laser is then tuned off the absorptive wavelength, usually by a few tenths of nanometers, to a wavelength outside of the absorptive spectrum and another pulse is emitted. Energy from both pulses is reflected back to a sensitive power detector, usually sensitive to the nano-watt or pico-watt level. The detector senses the reflected energy of both signals and the concentration or volume of the target constituent is determined by the relative intensities of the two signals.

Alternatively, the reflective characteristics of the target molecule can be used to detect relative volume. If the molecule is reflective at a known narrow-band wavelength, then a narrow-band pulse can be emitted from a single-mode laser and a sensitive power detector of the appropriate material can be used to detect reflected energy and the concentration of the molecule in a volume can be determined.

Polarization characteristics may also be used to identify the glucose molecules. Some materials absorb light in a particular polarization and re-emit it in another. For instance, a pulse of linearly polarized light may be absorbed by the compound and circularly polarized light re-emitted. Since the glucose molecules have a unique polarization characteristic as compared to the other constituent molecules in the blood, then the amount of glucose present can be identified.

A coherent system can be used to identify phase changes in the reflected pulse signal. Doppler techniques can also be useful because the glucose molecules are traveling through the bloodstream at either a faster or slower rate than surrounding constituents. The present invention is not limited to the optical detection methods described herein because the present invention can use any optical method of detecting blood glucose levels within the blood vessel.

Other types of compounds may also be tested for in the blood vessel and blood stream. For example, the present invention may test for hormones in the blood and then supply hormone therapy to a patient. Other substances that may be tested for can include molecules that are detectable using optical means. This may include vitamins, proteins and similar compounds.

Examples of the type of laser technology that can be used in the present invention will now be discussed. In order to more safely implement an intravenous monitor of glucose levels in the bloodstream, the laser delivery mechanism can deliver energy in very small amounts. Various materials and techniques can be implemented, depending on the wavelength, power, polarization, and coherence needs of the technique used. In the case of absorptive techniques, a wavelength of around 1.064 μm may be used. There are many commercially available lasers including one in a reasonably small package that emits up to 250 mW of single-mode, fiber-coupled, 1.064 μm energy, and is tunable over a wide range. Of course other wavelengths may be used as needed for a given target compound in the bloodstream.

The fiber used may be single-mode with a cladding diameter of around 50 μm that enables easy insertion into an IV. Intramodal dispersion that can lead to phase changes is minimal in single mode fibers, particularly over such short lengths of several meters. However, in embodiments where the dispersion is inhibitive to the detection technique, dispersion-flattened fibers are available. Polarization-maintaining fibers are also available in where the polarization characteristics are desired to be preserved.

When more fiber optic cable power is required, a fiber amplifier can be implemented. A 5-meter length of Yb:Silica fiber amplifier with a 400 μm, 0.4 NA octagonal cladding and a 30 μm, 0.06 NA core has been shown to produce over 5W of 1.064 μm energy. The fiber may be angle-polished on one end to suppress parasitic lasing due to internal reflections at the air/fiber interface.

For example, the fiber can be pumped from one end using a 30-Watt, 976 nm fiber coupled laser diode and seeded from the other end of the fiber so that the input seed signal and the pump laser are counter-propagating. An output power of approximately 5 Watts can be provided. Therefore, a fiber of much smaller cladding (on the order of 200 μm) may be used. Also, the amount of pump power from the laser diode can be reduced. The output light is still single-mode and polarization maintaining fiber is also available for the fiber amplifier so that none of the techniques mentioned herein are excluded.

Examples of detection techniques that can be used with the present invention will now be discussed. If direct detection of energy is to be used, then a fiber coupled to a receiver can be inserted into the IV along with the fiber transmitter. The reflected energy is coupled into the inserted receiver fiber via a molded lens at the end of the fiber. At the other end of the fiber (back at the control box), the fiber is coupled by a lens into a sensitive InGaAs photo-detector that proportionally converts the photons into current that is then input into the computation electronics. Alternatively, an avalanche photo-diode (APD) can be used to provide gain for the Optical-to-Electrical conversion.

In the case that polarization or phase information is desired to be detected, then a photo-detector may be inserted into the IV itself. Photo-detectors of diameters <30 μm have been manufactured for many applications. These detectors are mostly high-speed detectors frequently used in 10 GHz class telecommunications applications. The detector can be inserted into a hollow tube with a plastic formed lens at the end. The electrical signal is transmitted via a low-loss twisted-pair back to the electronics in the controller housing or box.

Any number of safety mechanisms can be incorporated into the computer hardware, software, and mechanical components of the system. These safety mechanisms can be setup to avoid potential problems and system malfunctions. Potential problems and method of overcoming these problems are discussed in the following list and include but are not limited to:
Thrombosis within the blood vessel. The fiber optic cable can be coated or embedded with an anticoagulant. In addition, there can be a minimum flow rate of the fluid through the catheter or an alarm 212 (FIG. 2) will alert the medical professional to “flush” the catheter.
Bending of the fiber optic cables. The fiber optic cables can be incorporated into the hollow tubing. Alternatively, the cables can be contained in their own casing that is rigid enough to prevent bending at acute angles but flexible enough to be practical in going from a container to the bedside of a patient.
Hypoglycemia. An auditory and/or visual alarm can be initiated when a glucose level in the blood is equal to or less than a reading level.
Hyperglycemia. An auditory and/or visual alarm can be initiated when a glucose level in the blood is greater than or equal to a reading level. Then the medical professional can give a bolus of the fluid (insulin mixture) through the interface.
Obstructed tubing preventing fluid flow. An alarm can be sounded and the pump can be temporarily disabled.
Human error. Visual prompts can assure that the proper concentration of insulin is in the bag of fluid. Other prompts or alarms can be included as defined by those skilled in the art.
Lack of information for medical professionals to provide appropriate medical care. Computer hardware and software can deliver all the information about the system either to the user interface or through a printer including all the blood glucose readings and insulin delivery or summaries thereof.

It should also be noted that the present invention may use an intravenous peripheral catheter of any gauge through which the fiber optic cable can enter the blood vessel to perform measurements. Further, the catheter can include an intravenous central catheter that contains any number of ports. The catheters can be inserted into the patient using the over the wire systems that are known to those skilled in the art. In addition, the fiber optic cable can be non-continuous and fully aligned with a connector that approximates the two free ends.

The present invention can include an embodiment of the container designed in any shape to hold equipment for the system. The container may include some or all of the following. A readout display can be provided using a liquid crystal display (LCD) or similar technology. In addition, a touch screen can be included that allows input of information by medical or other personnel.

A speaker 212 (FIG. 2) can be included to project an audible alarm along with a visual alarm displayed on the readout display. The visual alarm may display flashing and/or colored messages.

The container 218 can include computer hardware and software that facilitates computations based on a variety of data input from one or more sources, including the optics receiver. Computer hardware and software can be included that records and stores part or all of the data in the system. This data can also be displayed on a readout display or printed for further use.

The container 218 may also include a hinged 202 or spring-loaded door that opens for easy access to the system components. A depression can be formed in the casing of the container behind the door wherein a bag of insulin solution is placed or “hung” 210. The top of the bag may have a hole that is hung on a small post. The bottom of the bag has a nipple that sticks out away from the bag any number of centimeters and has on its end a pliable material that can be pierced manually with a sharp object. The nipple may also have a flange that fits into the depression for holding the bag firmly. The depression(s) in the casing of the container behind the door can be configured so that one end of the catheter tubing can rest after a sharp end of the catheter tubing is pierced into the nipple of the bag thereby allowing the insulin solution to flow from the bag through the tubing. The depression in the casing of the container behind the door can be configured wherein one end of the fiber optic cable can rest or come in direct contact with the laser device.

A line of pins can be used as a pump that sequentially presses on the tubing to direct a given volume of fluid down said tubing. The line of pins may receive directions from the computer hardware and software of the system. Other commonly known pumping mechanisms can be employed.

Example Optical Embodiment

An example embodiment of an optical detection system will now be described. The term optical used here generally means any electromagnetic energy that can be channeled through optical fibers. For example, optical energy can be infrared or within the visible spectrum or other useful spectrums.

Blood glucose concentrations can be determined by the application of coherent lidar techniques to an intravenous blood glucose monitor. The back-reflection system of the present embodiment allows for ease of implementation for an intravenous sensor due to the geometry of the sensor.

In optical detection sensors, light (laser energy in one embodiment) is transmitted through a scattering medium to a hard target. If the target is a rough surface (not a mirror), the energy is scattered into a full hemisphere. Some of this back-reflected energy is captured by the receive aperture and converted to an electrical signal. The received signal is large enough to create more electrons than the noise level of the receiver in order to obtain detection.

The specific response of the receiver to the signal power depends on the specific design details of the sensor. The signal power can depend on whether the receiver is direct detection or coherent detection. In direct detection, the return photons are measured directly with a photo-detector (usually a semiconductor diode that converts photons to electrons). One measure of system performance is the signal-to-noise ratio (SNR), which is a measure of the number of signal electrons that are higher than the electron noise floor of the sensor.

In coherent detection, the return signal photons are mixed with a local oscillator (LO) on the photo-detector. The LO is usually a small percentage of the transmit beam that is split from the primary beam prior to transmission. This LO can provide gain to the return signal. One measure of system performance for coherent detection is the carrier-to-noise ratio (CNR), which is also a measure of the number of signal electrons that are higher than the electron noise floor of the sensor.

All sensors (coherent and direct) have multiple noise sources including shot-noise from the signal, background light, detector dark current, electronics amplifier noise, thermal noise, and 1/f noise. In coherent detection systems, the LO may be increased until the LO shot noise dominates all other noise terms. The shot noise efficiency (ηₛₙ) is a measure of how much of the total noise is shot noise. In properly designed coherent detection systems, ηₛₙ can approach a value of 1.0.

Controlling factors such as shot noise lead this invention to minimize interference with the optical glucose signal and thus maximize the reliability and accuracy of the glucose measurement. Other important considerations in achieving this goal concern the following parameters:

1. Transmit and receiver optics efficiencies ¥ while these are generally conservative estimates, they are dependent upon the wavelength and bandwidth of the coatings, and the type of optic.

2. Transmit power ¥ the amount of transmitted power is dependent upon available sources at the wavelength of interest and what is considered to be a [symbol] safe [symbol] energy level to be transmitted through the patient [symbol] s blood.

3. Path length ¥ absorption and reflectivity are both highly dependent upon the path length of the optical signal. This would particularly effect DIAL detection.

4. Target reflectivity ¥ a conservative value is used based on experience in lidar detection systems.

5. Absorption ¥ absorption (or optical depth) of blood of different glucose concentration levels is needed.

6. Wavelength ¥ wavelengths that yield the best differential in compound absorption based on the light sources or filters used.

In another embodiment, optical detection of glucose concentrations can be achieved not as a result of backscattered energy but as absorbed and/or refracted light. In this sensor there are two fiber optic cables, one which is positioned flush or just outside the lumen of the catheter and another that extends beyond the first. The second cable is designed to receive the light energy that is emitted from the first cable. The second cable can contain a receiver diode. Alternately it can be shaped so that as it extends away from the first cable it makes a 180 degree turn and the entrance aperature for receiving the light is facing the exit aperature of the first cable. This configuration would be secured so that [symbol] direct line of sight [symbol] is guaranteed between the two cables. The light energy that travels between the two cables is altered by the glucose in the blood that flows between the two ends resulting in an optical reading by the second cable that is translated into an electrical signal for interpretation by the calculation module.

DIAL Embodiment

DIAL concentration detection is a relatively new technique for determining the abundance of a constituent molecule against background molecules. This technique uses a narrow-band, fast-switching laser to switch between an [symbol] on [symbol] wavelength and an [symbol] off [symbol] wavelength. The on wavelength is highly absorptive in the target molecule while the off wavelength is minimally absorptive (or nearly zero).

The concentration of the target molecule or compound is a function of the ratio of the backscattered intensities. The technique can be employed using either direct detection or heterodyne (coherent) mode. The direct detection method has the advantage of being more simple to implement, but is not as sensitive and, therefore, requires a detector with very low shot-noise, greater transmitter power, or larger absorption of the on wavelength (via a longer path length or larger absorption coefficient). Usually, a balance of these three conditions is required.

The main advantage of a heterodyne system is that a much greater sensitivity can be achieved. However, this increased sensitivity comes at the price of greater system complexity.

In order to use a DIAL system to detect glucose concentration levels, a determination is made of the differential optical depths of glucose between the on and off wavelengths, to calculate a plausible CNR off for the off wavelength, and determine the number of pulses to be sampled. The number of sampling pulses is determined by the required monitoring frequency. For example, 1×10⁶ samples can be used for a concentration monitoring rate of once per second. As glucose concentration and optical depth increase, the fractional error decreases exponentially. These errors can be improved upon by more sampling, a larger CNR, or a longer path length.

As compared to coherent detection systems, direct detection systems have the advantage of simplicity in system design, in that no local oscillator or mixer is used. However, these systems are more affected by noise, especially shot noise and speckle noise. High speckle diversity can be achieved by using a large detector that can view a large sampling of the reflected speckle simultaneously, or by taking a large sample of pulses whose phases are uncorrelated. If shot noise is then kept very low, direct detection can potentially perform better than coherent systems.

Pulsed systems have the advantage over continuous wave (CW) systems in that the higher peak powers gives greater assurance that the reflected signal will not be so weak as to be dominated by noise, although this is not likely in the absence of strong absorption.

In the case of direct detection systems, the scattering of pulses off of compounds causes the speckle to de-correlate in tens of nanoseconds due to the motion of the particles, whereas, in the case of a CW system against a hard target, speckle still de-correlates due to turbulence, but it may be a matter of microseconds or milliseconds.

One factor that should be considered in a pulsed system is that sampling is now dependent upon the pulse rate format (PRF) rather than the bandwidth of the receiver itself, as is the case in a CW system. A trade-off must be made between the PRF and the required output power of the receiver. A higher PRF results in more frequent sampling and, therefore, greater speckle diversity (in the case of direct detection), or (in the case of coherent detection) smaller measurement error. However, a higher PRF results in lower peak powers and can result in a low SNR due to a weak signal.

Polarimetry

Glucose tends to scatter light differently than background constituents, particularly water. A lidar-type system that can distinguish glucose from water and other blood constituents by detecting differences in the degree to which each type of molecule depolarizes incident light can assist in the accuracy of the glucose concentration measurement.

By placing filters between a light source and a polarization insensitive detector, the polarization state of any source can be determined. Physical interpretation of a normalized depolarization is simply that =0 suggests there is no depolarization, =1 indicates that all the light sent out returns in the orthogonal polarization. An =0.5 indicates that the return light has become completely depolarized (not necessarily randomly polarized, but that ½ of the return energy exists in each polarization state). This quantity is the defined depolarization ratio as used throughout the remainder of this report.

Because depolarization of scattered light can be caused by intimate interaction between the probe beam and morphological details of the scatterers, the strength of the depolarization signal may be modulated by particle absorption. By choosing a pair of wavelengths that are on and off a unique absorption feature, it is possible to probe the species-specific absorption cross section by observing the wavelength dependence of the depolarization.

Unlike differential absorption lidar (DIAL) techniques that probe absorption by observing the range derivative of the backscatter signal to deduce the extinction cross section of the medium, the depolarization ratio signal is very robust and independent of competing effects. Normalization of energy removes the influence of extinction to the scattering.

During polarimetry analysis, a good depolarization signal may be obtained when particles are comparable or larger in size than the wavelength. At wavelengths that are long when compared to particle size, the particles act as Rayleigh scatterers and there is little particle penetration or surface feature interaction with the probe light.

A strong differential depolarization backscatter signal may be obtained by differencing backscattered polarization information between wavelength regions. By measuring the differential depolarization ratio (or similarly, differential Mueller Matrix element ratios) between these two wavelengths, the depolarization signal may be observed relatively independent of size distribution similarities with background compounds. A strong correlation tends to exist between right-circular and left-circular depolarization between two wavelengths. The identical ratio for other constituents at the same wavelengths was near unity, indicative of low correlation.

Optical Signal Delivery System

The optical delivery system is based on coherent laser radar theory applied to this medical embodiment. A back-reflection system allows for ease of implementation for an intravenous sensor using a single intravenous needle. The laser in the system can also deliver appropriate wavelengths and energy levels to maximize absorption by the molecule of interest.

The specific response of the receiver to the signal power depends on the specific design details of the sensor (most importantly if it is direct detection or coherent detection). In direct detection, the return photons are measured directly with a photo-detector. For example, a semiconductor diode that converts photons to electrons may be used. The fundamental measure of system performance is the previously mentioned signal-to-noise ratio (SNR). In coherent detection the fundamental measure of system performance is the carrier-to-noise ratio (CNR). The CNR is an average (not instantaneous) measure of the receiver capability.

CNR is a strong function of aperture size. For a simple fiber delivery system, the aperture size is the diameter of the fiber core. Since coherent detection is based on mode mixing, single mode fibers must be used. However, single mode fibers have a core diameter of approximately 9 μm for 1.5 μm light. Therefore, an increased aperture size can improve CNR, primarily the use of a lens. For example, lensed fibers can be obtained that increase the aperture diameter up to 80 μm.

Another key parameter for CNR is the target surface reflectivity (ρπ). Most rough surfaces are well described by a lambertian surface, where the power of the back-reflected light decreases with angle from normal incidence (θ) by cosθ. Due to this cosθ dependence, lambertian sources reflect into π steradians for a hemisphere, as opposed to the normal 2 π steradians in a hemisphere. Therefore ρπ for a lambertian surface is defines as the power reflectivity (a number between 0 and 1) divided by π steradians.

A common conservative value for ρπ used in lidar modeling is 0.1/π, or 0.03183. For example, the very conservative value of 0.01/π, or 0.003183 may be used for the reflectivity of the vein walls.

Coherent detection provides many sensor advantages. With only ˜80 pW of signal power, the LO gain and shot noise limit of operation provided by coherent detection provide a good opportunity for retrieving the low back reflected signal level. The CNR equation allows initial system requirements to be defined, primarily by the aperture size. Even when using conservative efficiencies and reflectivities, a CNR>10 dB can be achieved, thus showing that a fiber based delivery system is effective for detecting the back-scattered signal from inside a vein.

Other primary sensor designs trade-offs include monostatic (single fiber for transmit and receive) and bistatic (separate fibers for transmit and receive). Other critical sensor parameters that can be modified are aperture size, fiber endface (flat, angled, lensed), and fiber mounting in the intravenous needle (parallel or angled). The transmission properties of blood and the reflective properties of the interior of veins may also be considered as these are key components in the optical path.

There are at least two architectures for optical sensors—monostatic (shared transmit/receive optics) and bistatic (the transmit optics are separated from the receive optics). The benefits and drawbacks of both types are discussed, along with several implementations.

Monostatic Sensors

Monostatic optical sensors simplify sensor alignment, reduce part count, and are smaller. For this application, a monostatic sensor is a single fiber in the needle that both transmits the laser pulses and receives the backscattered light. The main issue with monostatic coherent sensors is the [symbol] blind [symbol] time. Any back reflections from internal optics during the transmit pulse usually are orders of magnitude larger than the receive signal, thus the receiver is inoperable during the transmit pulse.

One method for addressing this blind zone is pulsing the transmit beam. For remote sensing lidars, this is acceptable since a 0.5 μs pulse equates to a 150 m “blind zone”. Since most lidars are sensitive out to several kilometers, pulsing is a viable solution. For an intravenous remote optical sensing, the path length can vary from 0.5 cm to ˜5 cm. A 0.5 cm path length equates to a 17 ps pulse.

Another method for addressing the blind zone in a fiber system is the use of a fiber circulator. A fiber circulator is a 3-port device where light enters port A and exits port B. Reflected light is collected in port B, and directed out port C. Current off-the-shelf fiber circulators provide ˜60 dB of isolation between port A and C. Therefore if 1 mW of power is input to port A, 1 nW of power would leak to port C. This can still be a problem for coherent detection systems. For a 9 μm diameter aperture (standard single mode fiber), the power received at the aperture was only ˜1.1 pW (1.1×10−12) However, external polarizers can be used to increase the crosstalk isolation.

Bistatic Sensors

Bistatic sensors mitigate the [symbol] blind [symbol] time and crosstalk issues with monostatic sensors. In one embodiment, a bistatic sensor uses two fibers in the intravenous needle, one to transmit the light and the other to receive the backscattered light. The issue with bistatic systems (besides increased part count and cost) is aligning the two fibers to optimize receiving the backscattered light.

In a bistatic, parallel fiber optic sensor, the return signal dropped as a function of fiber separation, and that the “depth of penetration” increases as the fiber separation distance increases. Both of these phenomenons are founded on the physics of the fiber field-of-view (FOV). The most signal will be received when both fibers have the same field of view on the same area for the longest amount of distance. This is why the “depth of penetration” increased for greater separations, because the transmit fiber beam and receive fiber FOV do not overlap until a greater distance from the fibers. This is one of the primary advantages of monostatic transceivers, the transmit beam and receive optics are by default the same FOV and overlapping. This beam overlap effect is captured in the mixing efficiency of the LO and signal beams. The absorption and scatter coefficients “have minimal impact on the depth of penetration”, meaning the glucose concentration should not affect the fundamental physical models for FOV overlap. One embodiment is based on Gaussian beam propagation.

In single mode fibers, the primary field that propagates though the core of the fiber can be well approximated by a Gaussian beam. Since standard Corning SMF-28 single mode fiber can have a core diameter of ˜9 μm, the diameter of the Gaussian beam is approximately 9 μm for λ>1260 nm. When the beam exits the fiber it propagates based on standard Gaussian beam propagation physics.

Standard fibers have cladding diameters of 125 μm. Therefore if two fibers were mounted parallel and side-by-side, the cores would be 125 μm apart and thus their FOV can just begin to overlap when the beams were at least 125 μm in diameter. Higher efficiency may occur when the beams are 250 μm in diameter or greater.

One advantage of a bistatic system is that the transmit and receive apertures can be of different size, whereas in monostatic they are by default the same size. The CNR increases for larger receiver apertures and a smaller aperture may be used to produce a larger beam size at a specified distance.

The 20 μm diameter fiber achieves a 250 μm beam diameter by 2.5 mm, therefore the transmit beam is well within the FOV of the 50 μm diameter receive fiber. At the anticipated working range of 10 mm, both fiber FOV are substantially overlapping and thus providing for a higher heterodyne mixing efficiency, ηh.

Receiver Aperture Diameter

Coherent detection is a single spatial mode detection. This means the receiver can detect that portion of the signal field matched to the amplitude profile and phase (the mode) of the local oscillator field. The mode of the received signal must therefore be maintained until it is mixed and detected with the local oscillator on a photo detector. For the bistatic fiber delivery system, the use of single mode fibers can be applied. In the NIR bands of interest (1260-1360 nm and 1525-1625 nm) standard single mode fibers have core diameters of approximately 9 μm.

A 9 μm receiver diameter aperture, therefore equates to a single shot CNR of ˜−3 dB. Whereas averaging can improve the CNR, the graph explicitly shows the advantage of using a larger receiver aperture to improve CNR. An optical magnifier (a lens) is needed to increase the receiver aperture while maintaining the single-mode quality of a 9 μm core. For an invasive sensor, the best approach is a lens attached to the end of the fiber to reduce the size and part count of components inserted in the body.

There are several different techniques for attaching lenses to fibers. The most direct is attaching a simple high-refractive index sapphire ball lens on the tip of a single mode fiber with heat, time, or UV curable optical grade epoxy. Since the lens is not limited in size, larger microlenses (even up to 1 mm) can be attached to the 125 μm diameter optical fiber (the cladding diameter).

The primary disadvantage with this approach is maintaining optical efficiency during alignment and the epoxy setting process. An additional disadvantage for an invasive fiber delivery system is risk of the lens detaching while inserted inside the circulatory system.

A different technique that reduces these two disadvantages is forming a simple microlens by heating the tip of the optical fiber. A heated cylindrical optical fiber produces a ball lens with an overall diameter on the order of three times the diameter of the original optical fiber. The resulting microlens is fused to the optical fiber, reducing the risk of lens detachment. The microlens refractive index is a weighted average between the core and cladding of the original optical fiber material (which for a single mode fiber will primarily be the refractive index of the cladding). Although the lens is now fused to the optical fiber, there is still a structural weak point at the junction due to the moment-arm created by a large diameter lens attached to a 125 μm fiber.

A different embodiment includes creating a “barrel lens” with a section of step-index multimode optical fiber (usually 50 μm or 62.5 μm core diameter) fused to the 9 μm diameter single mode fiber. Step index fibers have a constant index of refraction in the core, and a different constant index of refraction in the cladding. The difference in the refractive index causes total internal reflection at the boundary, thereby containing most of the power in the core.

A refractive surface is then created on the fiber endface by CO2 laser heating the endface. This refractive surface focuses the light captured by the ˜50 μm core down to the 9 μm single mode fiber core. The distinct advantage of this approach is the mechanical stability of the lens since the lens has the same profile as the optical fiber itself. Also, by using fusion splicing to attach the lens to the fiber, the lens-fiber interface now has the same structural integrity of the fiber itself. The primary disadvantage is the refractive lens must be relatively precisely formed to efficiently couple light from the 50 μm core to the 9 μm core.

To alleviate the inefficient lens forming step, a small portion of multimode gradient-index (GRIN) fiber can be fusion spliced to the end of the single mode fiber. The core of a GRIN fiber is created with a gradient-index material that acts as a lens throughout the fiber length. As light propagates down the fiber, it is focused at regular intervals along the fiber length. By fusion splicing the correct length of multimode GRIN fiber to the end of a single mode fiber, all light entering the multimode core within its acceptable NA will be focused onto the single mode fiber core. This approach alleviates the risk of creating a weak attachment point of the lens to the fiber. The lens-fiber interface is now as structurally sound as the fiber itself. In addition, the use of a multimode GRIN lens provides the larger aperture needed for high CNR and high efficiency coupling into the single mode fiber via a manufacturable process.

It is to be understood that the above-referenced arrangements are illustrative of the application for the principles of the present invention. It will be apparent to those of ordinary skill in the art that numerous modifications can be made without departing from the principles and concepts of the invention as set forth in the claims.

1. A method for determining intravenous blood levels of a target compound contained in a blood vessel of a patient, comprising the steps of:
detecting concentrations of the target compound within a patient's blood using a sensor device configured to optically test blood at a location within the blood vessel;
calculating a measured amount of a therapeutic compound to administer into the patient's bloodstream based on concentrations of the target compound detected in the blood; and
pumping a measured amount of therapeutic compound through a catheter into the patient's blood.

2. A method as in claim 1, wherein the step of detecting concentrations further comprises the step of detecting glucose concentrations within a patient's bloodstream using the sensor device.

3. A method as in claim 2, further comprising the step of pumping a measured amount of insulin into the patient's blood vessel in order to maintain blood glucose within a specified range.

4. A method as in claim 1, wherein the step of providing a sensor device further comprises the step of using an optical fiber supported by the catheter, wherein the optical fiber extends into the blood vessel.

5. A method as in claim 4, an electronic sensor for receiving optical radiation returned by the optical fiber.

6. A device for responsively delivering therapeutic compounds into a bloodstream, comprising:
an intravenous catheter inserted into a blood vessel of a patient;
a detector in optical communication with blood within the blood vessel via the intravenous catheter, the detector being configured to detect at least one target compound in the patient's bloodstream;
a processing unit configured to receive detector measurements from the detector and calculate a concentration of the target compound in the patent's bloodstream; and
a pump configured to deliver an amount of a therapeutic compound directly into the bloodstream as calculated by the processing unit.

7. A device as in claim 6, wherein the detector is an optical sensor using at least one optical fiber.

8. A device as in claim 7, wherein optical radiation is delivered into the bloodstream using at least one optical fiber.

9. A device as in claim 7, wherein at least a portion of the optical fiber is covered with anti-coagulant.

10. A device as in claim 6, wherein the detector can receive optical radiation to detect concentrations of a target compound in the patient's bloodstream.

11. A device as in claim 6, wherein the target compound is glucose and the therapeutic compound is insulin.

12. A device as in claim 6, wherein the intravenous catheter has a plurality of lumens.

13. A device as in claim 6, wherein the detector further includes a negative pressure pump to withdraw blood from the blood vessel and enable optical testing of the blood.

14. A device as in claim 13, further comprising a filter to separate serum from the blood to enable the serum to be subjected to optical testing.

15. A devices as in claim 6, wherein the calculation unit receives detector information in electrical form or optical form.

16. A device for responsively delivering therapeutic compounds into a bloodstream, comprising:
an intravenous catheter inserted into a blood vessel of a patient;
an optical fiber, supported by the intravenous catheter, the optical fiber being configured to deliver optical energy into the blood vessel;
an optical sensor coupled to the optical fiber and configured to receive optical backscatter radiation via the optical fiber in order to detect at least one target compound in the patient's bloodstream;
a processing unit configured to receive optical sensor measurements and to calculate the concentration of the target compound in the patent's bloodstream; and
a pump configured to deliver an amount of a therapeutic compound directly into the bloodstream as calculated by the processing unit.

17. A device as in claim 16, wherein the optical sensor further comprises a separate fiber optic cable configured to receive optical information from the target compound.

18. A device as in claim 16, wherein the optical fiber extends a distance from the intravenous catheter into the blood vessel.

19. A device as in claim 16, wherein at least a portion of the optical fiber is covered with anti-coagulant.

20. A device as in claim 16, further comprising a housing, including:
a laser source for generating optical radiation;
a receiver diode configured to sense optical radiation; and
a container for insulin that is in communication with the pump.

21. A device as in claim 16, further comprising an audible and visible alarm to notify personnel of problem situations selected from the group comprising: low blood sugar concentrations, high blood sugar concentrations, obstructed flow of fluid, inappropriate pump operation.
